(19) United States
(12) Patent Application Publication
(10) Pub. No.: US 2015/0087576 A1
(43) Pub. Date: Mar. 26, 2015

(54) COMPOSITIONS ASSOCIATED WITH AND METHODS OF MANAGING NEUTROPHIL MOVEMENT USING SERUM AMYLOID P (SAP)

(71) Applicant: THE TEXAS A&M UNIVERSITY SYSTEM, College Station, TX (US)

(72) Inventors: Richard Gomer, College Station, TX (US); Anu S. Maharjan, Houston, TX (US); David J. Roife, Houston, TX (US)

(21) Appl. No.: 14/365,321

(22) PCT Filed: Dec. 13, 2012
(86) PCT No.: PCT/US2012/069421

§ 371 Date: Jun. 13, 2014

Related U.S. Application Data
(60) Provisional application No. 61/570,445, filed on Dec. 14, 2011.

(51) Int. Cl. A61K 38/17

(52) U.S. Cl. CPC A61K 38/1716
USPC 514/1.5; 514/21.2

(57) ABSTRACT

The disclosure also relates to a method of reducing the number of neutrophils in a body region by administering a Serum Amyloid P (SAP) composition in an amount and for a time sufficient to suppress neutrophil movement into the body region. In particular, it relates to a method of reducing the number of neutrophils in a body region suffering from an acute injury or from a chronic or long-term disease. Further, the disclosure relates to a method of increasing the number of neutrophils in a body region by administering an anti-SAP antibody or material able to bind SAP. The disclosure further relates to compositions usable with these methods.

### STATEMENT OF GOVERNMENT INTEREST

At least a portion of this invention was developed using funding from the National Institutes of Health, Grant No. HL083029. The United States government has certain rights in the invention.

### TECHNICAL FIELD

The current disclosure relates to methods of regulating neutrophil movement in a patient or neutrophil numbers in a body region. In particular, it relates to methods of regulating neutrophil movement by regulating the amounts or activity of the protein Serum Amyloid P (“SAP”). According to one set of embodiments, neutrophil movement into a body region may be suppressed in a localized manner by providing SAP to a region and establishing a SAP gradient in the region. In another set of embodiments, neutrophil movement into a body region may be increased by depleting SAP or interfering with its function. In an alternative embodiment, increasing local concentration of SAP in a body region may facilitate neutrophil movement out of the region.

### BACKGROUND

### Serum Amyloid P

SAP, a member of the pentraxin family of proteins that include C-polysaccharide reactive protein (CRP) and pentraxin 3 (PTX3), is secreted by the liver and circulates in the blood as stable pentamers. The exact role of SAP is still unclear. SAP binds to sugar residues on the surface of bacteria leading to their opsonisation and engulfment. SAP also binds to free DNA and chromatin released by dead cells. Molecules bound by SAP are removed from extracellular regions due to the ability of SAP to bind Fcγ receptors. After receptor binding, SAP and any attached molecule are likely engulfed by the cell. Monocytes, macrophages, and neutrophils all have cell-surface Fcγ receptors. SAP also inhibits the differentiation of monocytes into fibroblast-like cells called fibrocytes.

### Tumor Necrosis Factor-Alpha (TNF-α)

TNF-α is a trimeric extracellular protein. It functions as a pleiotropic inflammatory cytokine Most organs of the body appear to be affected by TNF-α, and the cytokine serves a variety of functions, many of which are not yet fully understood. It possess both growth stimulating properties and growth inhibitory processes. TNF-α is produced by several types of cells, but especially by macrophages.

TNF-α plays an important role in the inflammatory response. It functions as an acute phase protein which initiates a cascade of cytokines and increases vascular permeability, thereby recruiting macrophages and neutrophils to a site of injury or infection. TNF-α secreted by macrophage causes blood clotting. TNF-α also encourages neutrophil migration and increases the adherence of neutrophils to a variety of extracellular matrices.

Movement of Neutrophils From the Blood into Body Regions

Infections or injuries to tissues such as the lungs cause the damaged cells to recruit immune cells, including neutrophils and monocytes, to the injury site. The transmigration of neutrophils to the site of injury or infection requires the interaction of neutrophils with endothelial cells and extracellular matrices. In blood vessels, neutrophils are generally quiescent, but after an injury or infection, neutrophils begin to tether and roll on the blood vessel using the selectin family of adhesion molecules such as CD62L, CD62P, and P-selectin glycoprotein ligand-1 (PSGL-1). These adhesion molecules interact with endothelial cell adhesion molecules such as E-selectin, P-selectin, and PSGL-1. Activated endothelial cells also interact with neutrophil glycoproteins such as CD44 and CD43 through E-selectin to slow neutrophil rolling. CD44 interacts with E-selectin and causes the redistribution of PSGL-1 or L-selectin on rolling neutrophils, which then promotes the tethering of neutrophils and slows down the rolling velocity. The slow neutrophil rolling allows neutrophils to sense signals such as IL-8, TNF-α, GM-CSF, or fMLP from damaged cells or infection, and activate integrin adhesion molecules such as CD11b and CD18. IL-8 is a neutrophil chemoattractant that can induce neutrophil degranulation and enhance neutrophil production of reactive oxygen species. TNF-α and GM-CSF increase neutrophil adherence, release of reactive oxygen species, and phagocytosis. fMLP resembles bacterial waste products, and activates neutrophil chemotaxis.

The upregulation of the adhesion molecules CD11b and CD18 let neutrophils interact with endothelial ligands such as intercellular adhesion molecule-1 (ICAM-1), which causes neutrophils to firmly adhere to the endothelium and move through the blood vessel into an injured site. Integrin molecules such as CD11b and CD18 can also bind to extracellular matrix components such as fibronectin, fibrinogen, laminin, and collagen, and this binding aids in the movement of neutrophils through extracellular matrices. Other integrin adhesion molecules such as CD61 facilitate leukocyte migration, but little is known about their roles in neutrophil migration. Once activated neutrophils are at injured sites, they can release reactive oxygen species and proteases, and then engulf bacteria and debris by phagocytosis.

In the normal resolution of wound healing, activated neutrophils undergo programmed cell death, which prevents the release of reactive oxygen species from the neutrophils, thereby preventing any cell damage in the surrounding tissue. Because activated neutrophils can damage surrounding cells, cytokines such as IL-4 and IL-10 inhibit excessive recruitment of neutrophils into the site of injury. IL-4 and IL-10 inhibit the production of IL-8 and the release of TNF-α and IL-1β, which in turn limits granulocyte (including neutrophil) accumulation and activation. Lipid mediators such as lipoxin A4 (LXA4) and lipoxin B4 (LXB4) inhibit neutrophil recruitment by reducing neutrophil adhesion to endothelial cells and vascular permeability. Other lipid mediators including D- and E-series resolvins and protectins also inhibit transendothelial migration of neutrophils.

Secreted pentraxin proteins such as pentraxin-3 (PTX3) and C-reactive protein (CRP) also limit neutrophil recruitment to a site of injury. PTX3 is a pentraxin that is produced and released by monocytes, dendritic cells, endothelial cells, and smooth muscle cells in response to inflammatory signals such as IL-1β, TNF-α, or TLR agonists. CRP is a pentraxin secreted into the blood by the liver as an acute phase protein in humans, and inhibits neutrophil adhesion and chemotaxis on activated endothelial cells. Neutrophils recognize the pentraxin family of proteins through Fcγ receptors. Neutrophils express high levels of FcγRII (CD32) and FcγRIII (CD16), and express low or undetectable levels of FcγRI (CD64). These receptors bind to the Fc portion of IgG immunoglobulins or pentraxin proteins such as PTX3, CRP, and SAP, and help in the opsonization and phagocytosis of bacteria or debris. However, reports regarding SAP binding to this and other proteins present on neutrophils are inconsistent.

### Effects on Neutrophils

Neutrophils are immune system cells found in the blood and elsewhere that are involved in the early stages of immune response and inflammation. Neutrophils are typically some of the first cells to leave the blood stream and enter the site of an injury or infection. Neutrophils begin to arrive at an infection or injury site within seconds to minutes, depending on blood circulation to the region. Once there, they release chemicals that further increase the immune or inflammatory response, for instance by recruiting more immune systems cells.

Serious medical conditions can result in patients who have low numbers of neutrophils in their blood stream or whose neutrophils do not appropriately leave the blood stream when needed. Such problems can occur due to genetic disorders, diseases, such as immunological disease, or the effects of immunosuppressant medications. These patients may be unable to respond appropriately to infection or injury, preventing proper wound healing or allowing infection to set in and become harder to combat later. Accordingly, a need exists for a method of increasing the ability of neutrophils to leave the blood stream and enter injury or infection sites in such patients.

Localized increases in the number of neutrophils in otherwise normal patients may also sometimes be useful. For instance, neutrophils may play a role in combating cancer. Cancer patients with otherwise normal neutrophil levels and activity might benefit from increased influx of neutrophils to a cancerous tumor or lesion, to the surrounding area, or even to the entire affected organ to help prevent metastasis. Currently, neutrophil-specific treatments are unavailable.

Serious medical conditions may also result when too many neutrophils enter a location in the body. Because the neutrophils have the ability to recruit additional immune system cells and to otherwise enhance the immune and inflammatory responses, they may cause harmful inflammation or damage from the immune system. There are two primary reasons for this to occur, either an immunological disorder or a severe injury.

An overabundance of neutrophils may result from an immunological disorder in any number of ways. For instance, some patients may simply produce too many neutrophils or may produce neutrophils that leave the blood stream too easily. Such patients may be at risk for an over-abundance of neutrophils in even a minor injury. More commonly, the patient may have an autoimmune disease, such as rheumatoid arthritis. Unusually high numbers of neutrophils are found in the joints of patients with rheumatoid arthritis, indicating their role in the development and progression of that disease. Unusually high and harmful numbers of neutrophils may be found in any organ or tissue affected by an autoimmune disease.

Another cause for a harmful overabundance of neutrophils is a severe injury. The body is often not able to address severe injuries and actually causes more harm in attempting to do so. For instance, severe injuries often result in a runaway effect, in which the body responds at higher and higher levels and in more and more ways until the negative effects of the over-response outweigh any positive ones.

For instance, there are over 200,000 cases of acute respiratory distress syndrome (ARDS) in the United States each year. ARDS can result from any severe injury to the lungs, such as infection or inhaling acidic materials, such as vomitus, but it most commonly results from smoke inhalation, particularly during house fires. During ARDS, a large number of lung cells are damaged, causing a rapid influx of neutrophils from the blood stream into the lungs. Once there, the neutrophils release reactive oxygen species and proteases that cause still further damage to lung cells, including the remaining healthy lung cells. These additional damages cause more neutrophils to enter the lungs, resulting in still more damage. Current treatments for ARDS are ineffective at halting this cycle of neutrophil influx and lung damage. As a result, 40% of all patients who develop ARDS die shortly thereafter.

Many ARDS patients do not sustain fatal levels of lung damage during the initial onslaught. As a result, if there were a treatment that, within a matter of a few hours, could deter neutrophils from entering the damaged lung or drive them back out of the damaged tissue, these patients could be saved.

Neutrophils also play a role in chronic obstructive pulmonary disease (COPD). Even though this disease is chronic, rather than acute in nature, there appears to be a constant influx of unhealthy levels of neutrophils into the lung tissue of COPD patients. Furthermore, the amount of neutrophils in the lungs correlates with the severity of the disease. Thus, if the number of neutrophils present in the lungs of COPD patients could be reduced, a corresponding improvement in symptoms is expected.

Treatments able to affect neutrophils entering the lung might also be useful in other situations. For instance, they may be able to help prevent any damaging effects of minor lung injuries, such as inflammation caused by minor air pollution.

Finally, treatments able to deter neutrophils from entering other inappropriate regions or able to drive them from those regions, such as arthritic joints, may be useful in treating other diseases, like rheumatoid arthritis

Other problems may result from too few neutrophils in a region of the body. The problems may be corrected or ameliorated by encouraging neutrophil influx.

### SUMMARY

This disclosure is based upon the finding of a new activity of SAP as an inhibitor of neutrophil adhesion. Various aspects of the disclosure may be carried out using a composition containing SAP, an anti-SAP antibody, or a material able to bind SAP.

One aspect of the present disclosure is a method of reducing the number of neutrophils in a body region comprising administering a serum amyloid P (SAP) composition in an amount and for a time sufficient to suppress neutrophil movement into the body region.

Another aspect of the present disclosure is a method of reducing the number of neutrophils in the lungs of a patient suffering from acute respiratory distress syndrome (ARDS) comprising administering a serum amyloid P (SAP) composition the patient in an amount and for a time sufficient to suppress neutrophil movement into the lungs.

An alternative aspect of the present disclosure is a method of reducing the number of neutrophils in the lungs of a patient suffering from acute lung injury (ALI) comprising administering a serum amyloid P (SAP) composition to the patient in an amount and for a time sufficient to suppress neutrophil movement into the lungs.

An additional aspect of the present disclosure is a method of treating a disease, wherein at least one of the indicators of the disease is the number of neutrophils in a body region of a patient, comprising administering a serum amyloid P (SAP) composition to the patient in an amount and for a time sufficient to suppress neutrophil movement into the body region.

### BRIEF DESCRIPTION OF THE DRAWINGS

A more complete understanding of the present embodiments and advantages thereof may be acquired by referring to the following description taken in conjunction with the accompanying drawings, which depict embodiments of the present disclosure.

FIG. 1A illustrates the normal movement of a neutrophil on a surface or in a tissue. Movement is indicated by dashed arrow.

FIG. 1B illustrates the inhibitory effect of SAP on the movement of a neutrophil on a surface or in a tissue. Lack of movement is indicated by lack of dashed arrow.

FIG. 2 illustrates the effect of human SAP on neutrophil spreading via microscopic images in which arrows indicate spread neutrophils. All of the images represent one of control, 10 μg/ml SAP, PBMC supernatant, or PBMC supernatant+10 μg/ml SAP. Bar is 20 μm.

FIGS. 3A-B also illustrate the effect of human SAP on neutrophil spreading. FIG. 3A shows microscopic images in which arrows indicate spread neutrophils. All of the images represent one of control, 10 μg/ml SAP, PBMC supernatant, or PBMC supernatant+10 μg/ml SAP. Bar is 20 μm. Images are representative of three separate experiments. FIG. 3B shows the percentage of spread neutrophils as compared to a BSA control for different experimental conditions. Values are mean+/−SEM, n=4

FIGS. 4A-4D illustrate the effect of human SAP on TNF-α-induced human neutrophil adhesion. ** indicates p<0.01, and *** indicates p<0.001 in FIGS. 4A-4C. FIG. 4A illustrates the effect on bovine serum albumin-coated culture plates. FIG. 4B illustrates the effect on plasma fibronectin-coated culture plates. FIG. 4C illustrates the effect on cellular fibronectin-coated culture plates. In FIG. 4D * indicates p<0.05 and * * * indicates p<0.001. FIG. 4D illustrates the effect on dried cellular fibronectin-coated culture plates. Values are mean+/−SEM, n=8.

FIG. 5 illustrates the effect of human SAP on TNF-α-induced murine neutrophil adhesion. * indicates p<0.05 and ** indicates p<0.01.

FIGS. 6A-6C illustrate the effect of SAP on mouse lungs treated with bleomycin. FIG. 6A illustrates the total number of cells obtained from bronchoalveolar lavage (BAL). FIG. 6B shows the total number of cells positive for the neutrophil cell surface marker, Ly6G in each of the experimental groups. The results are mean±SEM of the number of cells that positively stained for Ly6G (n=4 for control and mice treated with bleomycin and buffer, n=5 for mice treated with saline and buffer or mice treated with bleomycin and mouse SAP, and n=6 for mice treated with bleomycin and human SAP). FIG. 6C illustrates lung sections, after performing BAL, stained for Ly6G. Arrows indicate Ly6G-positive cells and the bars are 100 μm.

FIGS. 7A-7C also illustrate the effect of SAP on mouse lungs treated with bleomycin. FIG. 7A shows cells, after performing BAL, stained for Ly6G. Arrows indicate Ly6G-positive cells and bars are 20 nm. FIG. 11B illustrates lung section, after performing BAL, stained for Ly6G. Arrows indicate Ly6G-positive cells and bars are 100 nm. FIG. 7C shows the counts of cells positive for the neutrophil cell surface marker, Ly6G per mm in each of the above experimental groups Values are mean+/−SEM, n=3, * indicates p<0.05

FIGS. 8A-8C illustrate the effect of SAP on mouse lungs treated with an alternative dose of bleomycin than that illustrated in FIGS. 6A-6C. FIG. 8A illustrates the total number of cells obtained from bronchoalveolar lavage (BAL). FIG. 8B illustrates the number of cells positive for Ly6G. The results are mean±SEM of the number of cells that positively stained for Ly6G (n=4 for control, mice treated with bleomycin and buffer, or mice treated with bleomycin and human SAP, and n=5 for mice treated with saline and buffer). * indicates a significant difference with p<0.05 as determined by non-parametric Mann Whitney two-tailed t-tests. FIG. 8C illustrates lung sections, after performing BAL, stained for Ly6G. Arrows indicate Ly6G-positive cells and bars are 100 μm.

FIGS. 9A-9C also illustrate the effect of SAP on mouse lungs treated with bleomycin. FIG. 9A shows cells, after performing BAL, stained for Ly6G. Arrows indicate Ly6G-positive cells and bars are 20 nm. FIG. 9B illustrates lung section, after performing BAL, stained for Ly6G. Arrows indicate Ly6G-positive cells and bars are 20 nm. FIG. 9C shows the counts of cells positive for the neutrophil cell surface marker, Ly6G per mm in each of the above experimental groups Values are mean+/−SEM, n=3, * indicates p<0.05, *** indicates p<0.001.

FIGS. 10A-10C illustrate the effects of SAP on CD11b and CD45. FIG. 10A shows the number of CD11b-positive cells in the lungs of bleomycin-treated mice with or without the administration of SAP. FIG. 10B shows the number of CD45-positive cells in the lungs of bleomycin-treated mice with or without the administration of SAP. FIG. 10C illustrates lung section, after performing BAL, stained for CD45 or CD11b from mice in each of the experimental groups.

FIGS. 11A-11D present the effects of SAP on neutrophil surface receptors. FIG. 11A presents the results for neutrophils treated with IL-8, GM-CSF, or TNF-α in the presence or absence of SAP. The cells were then stained for CD11b. Values are mean+/−SEM, n=3. FIG. 11B presents the results for neutrophils treated with IL-8, GM-CSF, or TNF-α in the presence or absence of SAP. The cells were then stained for CD62L. Values are mean+/−SEM, n=3. FIG. 11C presents the results for neutrophils treated with IL-8, GM-CSF, or TNF-α in the presence or absence of SAP. The cells were then stained for CD32. The absence of an error bar indicates that he error was smaller then the line thickness. FIG. 11D presents the results for neutrophils treated with IL-8, GM-CSF, or TNF-α in the presence or absence of SAP. The cells were then stained for IgG1. The absence of an error bar indicates that he error was smaller then the line thickness.

FIGS. 12A-12B also present the effects of SAP on levels of neutrophil surface receptors. FIG. 12A presents results for human neutrophils treated as in FIG. 11A. The cells were then stained for CD11b or CD62L. Values are mean+/−SEM of percent median fluorescence intensity of the untreated CD11b-negative or CD62L-negative cells. (n=3 for all except UL-8 and IL-8+SAP, in which n=2.) FIG. 12B presents results for human neutrophils treated as in FIG. 11C and then stained for CD32 or IgG1. Values are mean+/−SEM of percent median fluorescence intensity of the untreated CD32-negative or mouse IgG!-positive cells.

FIG. 13 illustrates the effects of SAP on neutrophil surface receptors CD18, CD61 and CD44 also incubated in the presence or absence of TNF-α or fMLP. Values are mean+/−SEM of median fluorescence intensity of positively stained control cells (n=3 for CD18 and CD61, n=2 for CD44).

FIGS. 14A-D illustrate the effects of SAP on production of hydrogen peroxide in neutrophils. FIG. 14A shows the fluorescence intensity of scopoletin converted to hydrogen peroxide production in neutrophils pre-treated with or without SAP. FIG. 14B presents the same data for neutrophils pre-treated with or without TNF-α. FIG. 14C presents the same data for neutrophils pre-treated with or without PDBu. FIG. 14D presents the same data for neutrophils pre-treated with or without PMA. All figures represent one of three separate experiments or, in the case of PDBu, two separate experiments.

FIG. 15 shows the effects of SAP on neutrophil migration. Percent migration in the presence or absence of SAP and fMLP, or both is shown. The results are mean+/−SEM of migrated neutrophils (n=4).

FIG. 16 illustrates the effects of SAP on neutrophil apoptosis. Annexin V levels for neutrophils treated with GM-CSF or TNF-α in the present or absence of SAP are presented. The results are mean+/−SEM of percent positive cells (n=3).

### DETAILED DESCRIPTION

Unless otherwise specified, when “SAP” or “serum amyloid P” is used herein, it will be understood to include portions of SAP. The current disclosure relates to SAP compositions and methods of use thereof for regulating neutrophil movement into one or more body regions and for limiting the number of neutrophils in one or more body regions by administering SAP to the region, and, in some embodiments, to an administration site in the region, in an amount and for a time sufficient to have the desired effect. The disclosure also relates to methods of preventing, alleviating, or avoiding one or more symptoms or complications of an acute injury or chronic or long-term disease characterized by excess neutrophils in one or more body regions. For purposes of this disclosure, a body region may include one or more tissues.

The current disclosure also relates to anti-SAP antibody compositions and methods for use thereof to increase neutrophil movement into a body region, or facilitate the retention of neutrophils in that body region.

### Effects of SAP

One feature of the present disclosure is the effect of SAP upon neutrophils. In some embodiments, SAP may limit the influx of neutrophils into a certain region or tissue. This may be beneficial in efforts to limit an inflammation response encouraged by neutrophils or other damage resulting from the presence of excessive neutrophils in the body region. For example, in ARDS, neutrophils may cause undesired damage through escalation of an inflammation response. The inhibition of the influx of neutrophils into lung tissue may assist in the treatment of ARDS.

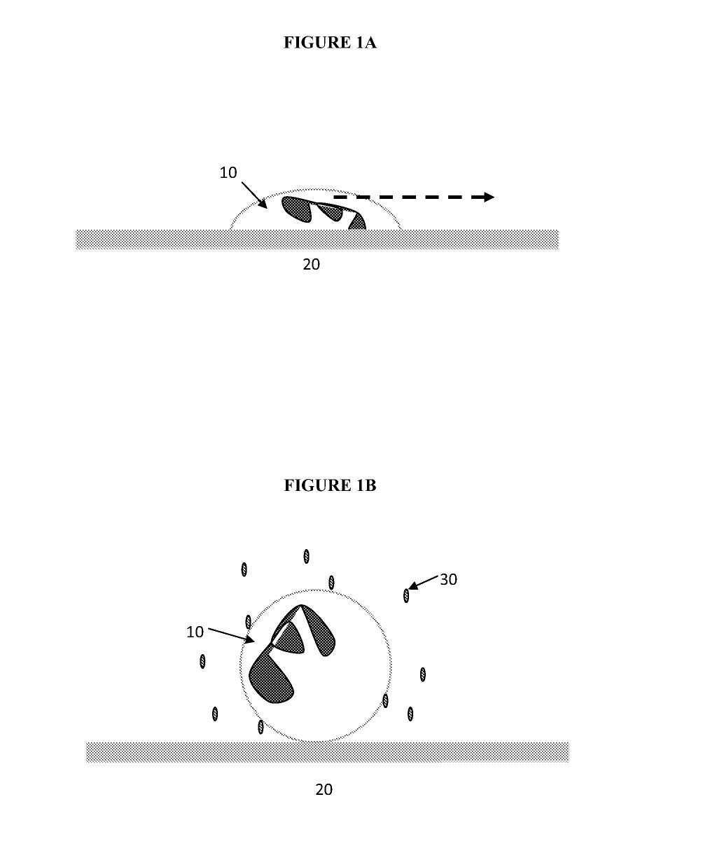

Neutrophils 10 normally move along a surface 20 as shown in FIG. 1A. The presence of SAP 30 in a region suppresses neutrophil movement, as shown in FIG. 1B.

Without limiting the present disclosure, SAP may exert its effects on neutrophils by affecting transmigration of neutrophils into a body region through its effects on interaction of neutrophils with endothelial cells and extracellular matrices.

Additionally, an alternative feature of the present disclosure is the effect of SAP upon neutrophil spreading. For example, SAP may inhibit neutrophil spreading. This may be by direct action of SAP upon the neutrophils. This may also cause a rounded morphology in neutrophils. Environmental components such as bacteria and cell debris may elicit the release of factors that signal neutrophil spreading, adherence, and migration to sites of infection or injury. SAP may inhibit spreading of neutrophils in the presence of such factors. An example of this feature is described in further detail in Example 1 below.

Another feature of the present disclosure is the effect of SAP upon neutrophil binding facilitated by TNF-α. For example, SAP inhibits neutrophil adhesion induced by TNF-α. In some embodiments, SAP may inhibit the binding of neutrophils to bovine serum albumin (BSA), cellular fibronectin, plasma fibronectin, or other matrix materials as are known in the art. This may be observed for neutrophils pretreated with SAP, or for neutrophils treated with SAP after exposure to the matrix. Additionally, this may occur once a matrix has been dried and SAP may still produce the inhibitory effect on the binding of neutrophils. An example of this feature is described in greater detail in Example 2 below.

Without being limited to a single mode of action, SAP appears to affect neutrophil influx by affecting their adhesion to extracellular matrix components and spreading. SAP does not, however, appear to affect the neutrophil adhesion molecules CD11b, CD62L, CD18, or CD44. SAP further does not appear to affect production of hydrogen peroxide by resting or stimulated neutrophils or fMLP-induced neutrophil migration. These effects are described in further detail in Example 7 below.

### SAP Compositions

Methods of the current disclosure may use compositions including SAP in any formulation sufficient to allow its administration to a region of the body. These specific formulations may be compositions of the disclosure. The SAP contained in such compositions may be human SAP, or it may be a non-human form. It may, in particular, be a mammalian form. It may be from a human or animal source, or it may be recombinant. The SAP may be full-length or it may be portions thereof. The sequence of SAP was determined by Woo et al. Patricia Woo, Julie R Korenberg, and Alexander S. Whitehead, Characterization of Genomic and Complementary DNA Sequence of Human C-reactive Protein, and Comparison with the Complementary DNA Sequence of Serum Amyloid P Component, 260 THE J. OF BIOLOGICAL CHEMISTRY 13384, 13387 (Oct. 25, 1985), which reference is incorporated herein by reference in its entirety.

Formulations of SAP may include any formulation sufficient to preserve the inhibitory effects of the protein on neutrophil adhesion. Different formulations may be useful for different variants of the protein, such as full-length protein or portions thereof, depending on different stabilities. The formulation may also be tailored to the intended use. For instance, the formulation may be suitable for administration via topical administration, injection or inhalation. An inhalable formulation may be suitable for use in a nebulizer. Topical formulations may include any suitable cream, ointment, emollient, gel, foam, or transdermal patch as a carrier.

The form of SAP or the formulation may be tailored to retain the SAP in a localized fashion. For instance, the formulation may contain materials designed to prevent SAP from entering the blood stream. The SAP itself may be glycosylated or it may have non-naturally occurring materials, such as polymers, bound to deter its diffusion into the blood stream or away from the site of administration. In particular, biodegradable and non-immunogenic polymers such as polyethylene glycol or poly (amino acids) may be attached to the SAP. Any other materials bound to SAP may be bound to regions that do not interfere with its effect on neutrophil adhesion.

Formulations of SAP may also include a pharmaceutically acceptable carrier, in particular a carrier suitable for the intended mode of administration, or salts, buffers, or preservatives. In particular, the pharmaceutically acceptable carrier may be tailored to allow SAP to retain an active conformation and to avoid degradation.

SAP formulations may include other pharmaceutically effective materials, such as materials able to repel or destroy neutrophils or other immune cells, or other materials able to otherwise induce short or long term beneficial effects in the affected tissue. For example, SAP formulations may include dipeptidyl peptidase-IV (“DPPIV”), which also affects neutrophils as described in U.S. patent application Ser. No. 13/571,841, filed Aug. 10, 2012 and titled “Compositions and Methods for Regulating Neutrophil Movement and Neutrophil Numbers in a Body Region,” incorporated by reference in material part herein. SAP compositions may also include steroids, non-steroid anti-inflammatory drugs (NSAID), or combinations thereof. SAP compositions may include biologically compatible polymers.

### Use of SAP

SAP, in one of the compositions described above, may be administered locally to a region of the body, for example at one or more administration sites, in an amount and for a time sufficient to suppress neutrophil movement into that region or to enhance movement of neutrophils out of that region. This results in a decreased number of neutrophils in the region as compared to prior to administration of SAP or as compared to the number of neutrophils that would be present absent the administration of SAP. The decreased number of neutrophils may prevent, alleviate, or avoid one or more symptoms of complications of an acute injury or chronic or long-term disease characterized by an excess of neutrophils.

The region to which a SAP composition is administered may be any body region with an unwanted number of neutrophils and the condition treated may be any acute injury or long-term or chronic disease in which an undesirable number of neutrophils are present.

In some embodiments of the present disclosure, SAP may be used to treat ARDS, as neutrophils appear in the lungs earlier than fibrocytes. The cells obtained from the bronchoalveolar lavage (BAL) of patients with acute respiratory distress syndrome (ARDS) show a significant increase in the number of neutrophils in the lungs compared to the cells obtained from the BAL of normal, healthy individuals. Cells obtained from the BAL of ARDS patients consist of 72% neutrophils, 25% alveolar macrophages, and 2% lymphocytes, while cells obtained from the BAL of normal, healthy individuals consist of 0% neutrophils, 91% alveolar macrophages, and 9% lymphocytes. After some time, fibrocytes can then be detected in the BAL of ARDS patients, and patients with the similar, but less severe, syndrome called acute lung injury (ALI).

Animal models of ARDS and fibrosis show different timings in the migration of innate immune cells such as neutrophils and monocytes after injury. In animal models of ARDS, the number of neutrophils in the lung increases as early as 4 hours after the injury and peaks at day 3. Macrophages and fibroblasts appear in these injured sites after 3-5 days, and the number of neutrophils remains elevated at these time points. There are neutrophils present in the injured sites as late as 2 weeks, and the numbers decline after 4 weeks. Repair cells such as fibrocytes appear at day 2, well, after neutrophils first appear, and do not reach a maximal concentration until day 8. Fibrocytes persist through day 21.

In some embodiments, SAP may be used to treat acute lung injury (ALI). One of the initial indications of ALI is migration of neutrophils into lung tissue. The use of SAP to decrease the number of neutrophils may be an effective treatment of ALI. This may occur by inhibiting the movement of neutrophils into the lungs. Additionally, this may occur by limiting an immune response, an inflammation response, or a combination of the two. Although the pathogenesis of ALI and ARDS involve both inflammation and fibrosis, the two occur at different time intervals and each stage must be treated as different disease processes. An accepted animal model of ALI involves intratracheal instillation of bleomycin, and this produces early histological events similar to those of ARDS in humans. Even though this eventually results in fibrosis, the two processes are separated by a definite time interval, and a distinction between the early inflammatory phase and the late fibrotic phase is appreciated by histological and gene expression data. Furthermore, different interventions at different time points have been shown to affect the course of the disease in independent ways. This early inflammatory stage of lung injury has been found to be mediated by an early accumulation of neutrophils.

SAP compositions may also be administered to treat long-term or chronic diseases of the lungs, such as COPD, asthma, or cystic fibrosis. As explained above, patients with COPD have a constant influx of unhealthy levels of neutrophils into their lungs. Neutrophils also play a role in asthma, especially in patients with chronic or severe asthma and asthma resistant to corticosteroids. In addition, it appears that there are increased numbers of neutrophils in non-allergic forms of asthma, such as those induced by air pollution, infection, and obesity. Therefore, in these forms of asthma where conventional therapies are not effective, treatment with SAP may be benefical.

Cystic fibrosis (CF) is a single-gene disorder caused by mutations in a chloride channel (CFTR). Lung disease is a major problem for patients with CF, leading to persistent bacterial infection and exaggerated inflammatory responses with elevated numbers of neutrophils. These neutrophils appear to be responsive for the release of proteases (enzymes) that damage the lung tissues. Therefore, treatment with SAP may be beneficial in these situations.

In another example, the body region treated with SAP may be a joint and the condition may be rheumatoid arthritis, which is also characterized by excessive numbers of neutrophils.

In addition to treatments associated with the lungs and joint, the features of the present disclosure may also be beneficial in the treatment of other diseases. In some embodiments, the indication being treated may include neutrophil-induced tissue damage. By way of example, the following diseases and regions of the body may be treated within the scope of the present disclosure.

In some embodiments, the present disclosure may be used to treat traumatic brain injury. In addition to the initial insult of the traumatic event to the brain parenchyma, there is a significant amount of secondary damage produced by secondary influx of inflammatory cells and edema. Neutrophil accumulation in the injured brain tissue is an early event seen after traumatic brain injury. Multiple animal models have proposed a role for halting this neutrophil infiltration as a method for limiting secondary damage to injured brain tissue. One of these methods, hypothermia, has been shown to both decrease neutrophil accumulation in an animal model and produce significantly improved clinical outcomes on human patients. Therefore, controlling neutrophil influx may be an effective treatment for brain injury.

The acute phase of tissue transplant rejection may also be treated within the scope of the present disclosure. Acute tissue transplant rejection remains a significant burden in transplant medicine despite improved methods to aid pre-transplant compatibility screening and improved post-transplant immunosuppressive drugs. This rejection is mediated by both allo-antigen primed T-cells that infiltrate the tissue and attract other inflammatory cells such as neutrophils as an effector cell to produce tissue damage and antibody deposition that activates the complement system which also attracts and activates neutrophils. Massive early neutrophil influx into the transplanted tissue has been demonstrated in allografts undergoing acute rejection in both human patients and in animal models of cardiac, liver, kidney, lung, small bowel, and pancreas transplants. Attenuating this inflammatory neutrophilic response may prevent much of the damage done during the acute phase of rejection.

Neutrophil influx into the liver causes liver damage in alcohol-induced neutrophilic steatohepatitis and after acetaminophen overdose. Therefore, these diseases may be treated within the scope of the present disclosure.

In the kidney, neutrophil influx causes tissue damage in acute glomerulonephritis and/or renal inflammation. Acute glomerular injury has been shown to be incited by circulating immune complexes that deposit in the glomerular basement membrane. These immune complexes activate the complement cascade, which then attracts inflammatory cells that further damage the glomerular basement membrane while trying to break down these immune complexes. Neutrophils have been shown to be an important effector cell causing damage to the glomerulus in various forms of acute glomerulonephritis through production of oxidants, cytokines, and chemokines These can include immune-complex type acute glomerulonephritides such as post-streptococcal glomerulonephritis, Goodpasture syndrome, rapidly progressive glomerulonephritis, membranoproliferative glomerulonephritis, IgA nephropathy, as well as glomerulonephritis caused by antineutrophil cytoplasmic antibody (ANCA)-associated vasculitis (AAV). The early stages of acute glomerulonephritis consist of mainly an inflammatory reaction consisting of neutrophils, with no atrophic or fibrotic changes seen in late stages of chronic glomerulonephritis. This suggests that treating these diseases early in their course with an agent that can prevent neutrophil influx and activation may ameliorate the damage caused by neutrophils during the natural history of the disease. The compositions of the present disclosure may be used to treat early stages of acute glomerulonepthritis.

The compositions of the present disclosure may be used to treat postoperative ileus. Postoperative ileus may be described as a reduction of gastrointestinal motility following an abdominal surgery. It is a significant medical problem that causes patients prolonged discomfort, and it is one of the most common reasons for delayed discharge from the hospital after abdominal surgery. There has not been much progress towards the treatment or prevention of this consequence of surgery, other than the recent adoption of laparoscopic surgery, which has shown to significantly attenuate postoperative ileus, presumably due to a reduced amount of trauma to intra-abdominal tissues. In animal models, it has been shown that trauma to the gastro-intestinal tract is evidenced on a cellular level by an early neutrophilic infiltrate. The invading neutrophils secrete many pro-inflammatory cytokines and cytotoxic substances that contribute to gastrointestinal tract dysmotility. Therefore preventing this early influx of neutrophils may lessen the amount of postoperative ileus, allowing patients to avoid unnecessary suffering and hospitals to save a tremendous amount of resources.

Chronic and long-term intestinal diseases, such as Crohn's disease and ulcerative colitis also involve the influx of neutrophils into an intestinal tissue. The present disclosure includes periodic administration of SAP to reduce the number of neutrophils in such tissues and thereby to also reduce one or more symptoms of these diseases.

In some embodiments, the compositions of the present disclosure may be used to treat acute pancreatitis. Acute pancreatitis remains one of the most frequent causes for hospitalization for gastrointestinal related problems, with over 250,000 admissions and costs of over $2 billion per year. Acute pancreatitis can have a wide spectrum of presentations, from mild discomfort to surgical emergencies and multi organ failure resulting in death. From clinical data, it has been demonstrated that pro-inflammatory mediators in the blood of acute pancreatitis patients correlate with the severity of that episode of acute pancreatitis. One of these mediators, PMN-elastase, which is secreted by neutrophils, was found to be one of the most useful indicators of severity. Animal models of acute pancreatitis have also exhibited an early influx of neutrophils into the pancreatic tissue, and that inhibiting this neutrophilic response by ways of neutrophil depletion and neutrophilic receptor blockade have lessened the severity of the disease course of both the pancreatitis itself and associated distant organ damage. Compositions of the present disclosure may also be used to treat chronic pancreatitis.

In the skin, neutrophil influx causes tissue damage in Sweet's syndrome/acute febrile neutrophilic dermatosis, rheumatoid neutrophilic dermatitis, pyoderma gangrenosum, subcorneal pustular dermatosis, Behcet's syndrome, palmoplantar pustulosis, neutrophilic eccrine hidradenitis, bowel-associated dermatosis-arthritis syndrome, and synovitis-acne-pustulosis-hyperostosis osteomyelitis (SAPHO) syndrome.

Neutrophil influx may also cause tissue damage in systemic septic shock, and treatment thereof is within the scope of the present disclosure.

Gout and other crystal-induced arthropathies, which are classis inflammatory diseases where neutrophils cause a considerable amount of damage, may also be treated with formulations of the present disclosure.

Additionally, reperfusion injury, such as pressure ulcers, diabetic foot ulcers, myocardial infarction, stroke, ischemic brain injury, and ischemic bowel disease, may be treated with formulations of the present disclosure. Reperfusion injury occurs following the return of blood flow to a tissue. During the period that blood flow is restricted or stopped, the lack of oxygen (ischemia) leads to cell damage and necrosis, which when blood flow returns (reperfusion) leads to the influx of immune cells, led by neutrophils. The neutrophils are then activated by the presence of the dead and dying cells, leading to inflammation. The administration of SAP to the local area may inhibit the movement of neutrophils into the site of reperfusion injury, thus reducing tissue damage and preventing further influx of immune cells.

The SAP composition supplied to a body region may include any SAP composition described herein or known in the art. The body region to which SAP is supplied may be any region containing an unwanted number of neutrophils.

The SAP may be supplied to the region in any suitable manner. For instance, in patients with rheumatoid arthritis, it may be injected into an administration site in an affected joint via fine needles. For a patient with danger of an abnormally strong response to a minor injury, it may be applied topically to a minor wound. For patients with ARDS, lung irritation, or any other medical problem associated with excess neutrophils in the lungs, it may be provided via inhalation, for instance using a nebulizer. For patients with systemic neutrophil problems, an injection of SAP may be provided. For patients with bowel-based neutrophil indications, an ingestible form may be used. In other embodiments, SAP may be supplied systemically.

In some embodiments, involving acute neutrophil influx, SAP may first be administered within 30 minutes, within 45 minutes, or within 60 minutes of a severe injury that leads to an acute influx of neutrophils. In other embodiments, SAP may first be administered up to within 24 hours or even within 48 hours after such an injury. In many embodiments addressing acute neutrophil influx, treatment is ideally begun as soon as possible after the injury, however, not all patients or the cause of their injury are discovered until some time has passed. Treatment may be provided continuously or at intervals until the danger of neutrophil influx passes. For example, in some embodiments, treatment may be provided continuously or at intervals for at least 24 hours, at least 48 hours, at least 72 hours, at least 96 hours, or at least 120 hours. One may determine when it is appropriate to cease treatments by observing when neutrophil influx, or the symptoms of neutrophil influx, no longer occur. In embodiments in which treatments are administered at intervals, intervals may be spaced such that substantial neutrophil influx does not resume between treatments or so that a sufficient concentration of SAP is maintained in the body region. For example, treatments may be repeated at least every 30 minutes, at least every 60 minutes, at least every 120 minutes, or at least every 24 hours. The time between intervals may increase as time after the injury increases.

In treatment of chronic or long-term diseases, such as rheumatoid arthritis or COPD, treatment may be administered periodically at intervals sufficient to decrease the number of neutrophils in the affected body region. For example, treatment may be administered at least every 24 hours, every 48 hours, every 72 hours, every 96 hours, every 120 hours, every week, or every two weeks.

The amount of SAP administered may vary depending on the location of administration, the mode of administration, whether an acute injury or chronic or long-term disease is being addressed, the planned treatment regiment, including dosing intervals, the severity of the injury or disease, and whether SAP is being administered for its effects on neutrophil responsiveness to TNF-α.

### SAP Reduction

As explained in the background above, some patients may benefit from increased neutrophil movement into or retention in a location. Such patients therefore may benefit from administration of a SAP-inactivator or inhibitor to that region or removal of SAP from that region. An inactivator or inhibitor may neutralize the ability of SAP to inhibit neutrophil influx. One such inactivator may be an anti-SAP antibody. Anti-SAP antibodies may include antibody fragments and may be provided in any formulation, such as any pharmaceutically acceptable carrier. Due to the role of SAP's activity in the regulation of wound healing and scar tissue formation, as well as its role as an inhibitor of neutrophil adhesion, in most instances anti-SAP antibodies may be administered in a localized manner, for instance by localized injection into a tumor or in the form of a topical wound dressing.

SAP may also be removed from a location by administration of a compound known to bind to SAP, such as R-1-[6-[R-2-carboxy-pyrrolidin-1-yl]-6-oxo-hexanoyl]pyrrolidine-2-carboxylic acid (CPHPC), certain agarose moieties often found in high EEO agarose, the 4,6-pyruvate acetyl of beta-D-galactopyranose, and phosphoethanolamine. The removal of SAP from wounds is known to facilitate wound healing. Thus, such compositions may provide two benefits to any wound that benefits from increased neutrophil influx. Such compositions may also be administered locally, for example in a topical wound dressing.

### EXAMPLES

The following examples illustrate certain embodiments of the disclosure. They are not intended as a description in full detail of every aspect of the disclosure and should not be interpreted as such.

### Example 1

### Detection of SAP's Effect on Neutrophil Spreading

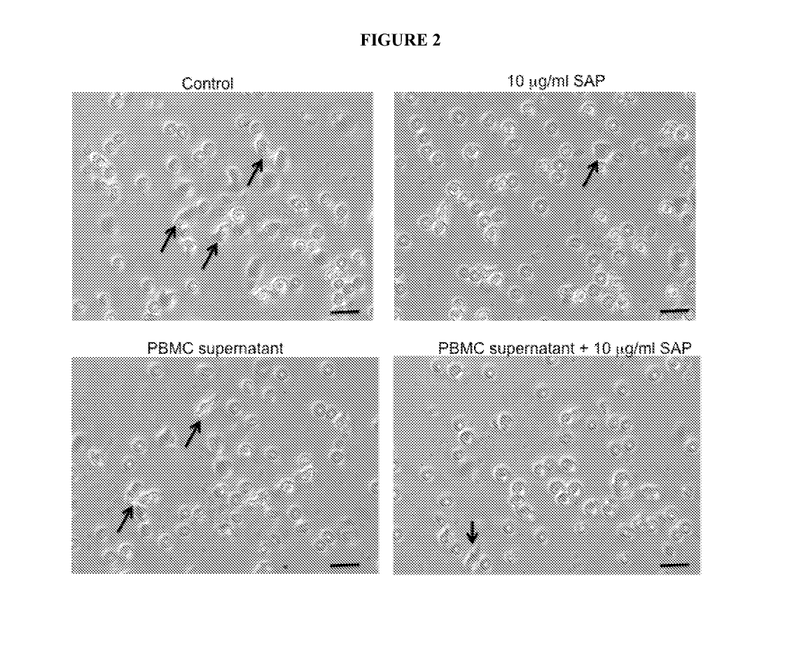

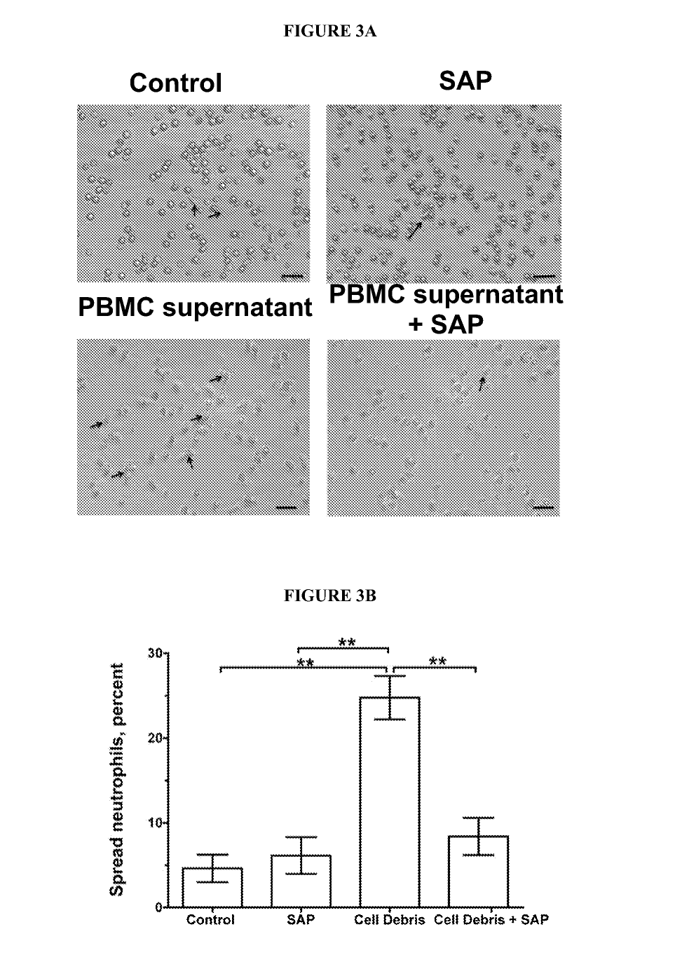

Neutrophil spreading allows neutrophils to polarize and migrate to the site of an injury. To determine the effect of SAP on neutrophil spreading, human peripheral blood mononuclear cells (PBMC) were isolated and incubated in the presence of cell debris for 7 days at 37° C. On the 7th day, the conditioned media was removed. Human neutrophils were incubated in the presence or absence of 10 μg/ml human SAP (hSAP), PBMC conditioned media, or the combination of PBMC conditioned media and SAP for 1 hour at 37° C. Fields of neutrophils were photographed using a phase-contrast microscope with a 20× objective at 1 hour. As can be seen in FIG. 2, the presence of SAP limited the presence of spread neutrophils. Results were repeated in a similar test in which neutrophils were incubated in the presence or absence of 20 μg/ml hSAP. The results from this test, presented in FIG. 3, show that although PBMC supernatant increased the numbers of spreading neutrophils, the addition of SAP inhibited the PBMC supernatant-induced neutrophil spreading.

### Example 2

### SAP Inhibition of Neutrophil Adhesion Induced by TNF-α

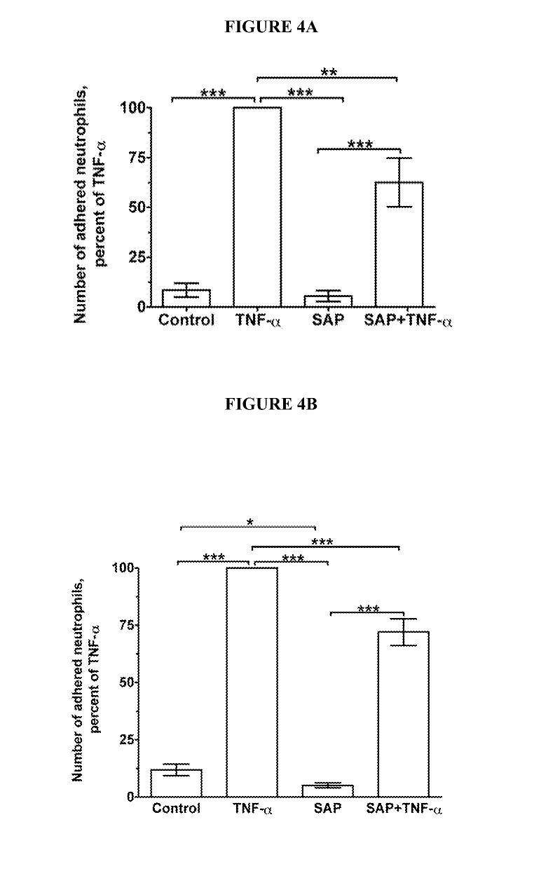

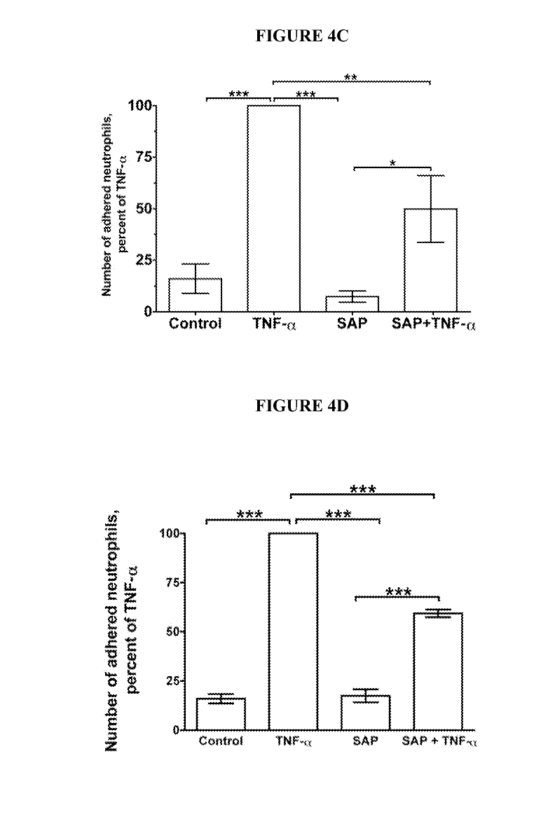

96-well tissue culture plates were blocked with 2% BSA-PBS for 2 hours at room temperature. The plates were washed in PBS, and once with 2% BSA-RPMI. Human neutrophils were pretreated with or without 30 μg/ml hSAP for 30 minutes at 37° C. 100 μl of 1×10⁶ cell/ml neutrophils were seeded on the plate, and 1 μl of 10 μg/ml recombinant human-TNF-α was added to the neutrophils for another 30 minutes at 37° C. After removing the media and washing the plates, the plates were air-dried, fixed, stained, and the number of neutrophils that remained in the wells was counted (n=4). FIG. 4A illustrates the number of neutrophils adhered to the plate.

96-well tissue culture plates were pre-coated with 20 μg/ml plasma fibronectin and then blocked with 2% BSA-PBS for 2 hours at room temperature. Neutrophils were incubated in the presence or absence of 30 μg/m1 SAP or 100 ng/ml TNF-α and counted as described above (n=23). FIG. 4B illustrates the number of neutrophils adhered to the plasma fibronectin.

96-well tissue culture plates were pre-coated with 20 μg/ml cellular fibronectin and then blocked with 2% BSA-PBS for 2 hours at room temperature. Neutrophils were incubated in the presence or absence of 30 μg/ml SAP or 100 ng/ml TNF-α and counted as described above (n=5). FIG. 4C illustrates the number of neutrophils adhered to the cellular fibronectin.

96-well tissue culture plates were pre-coated with 20 μg/ml cellular fibronectin and then blocked with 2% BSA-PBS for 2 hours at room temperature. Plates were then air-dryed. Neutrophils were incubated in the presence or absence of 30 μg/ml SAP or 100 ng/ml TNF-α and counted as described above (n=5). FIG. 4D illustrates the number of neutrophils adhered to the cellular fibronectin.

As shown in FIGS. 4A-4D, when the number of neutrophils treated with SAP, SAP and TNF-α, or control was normalized to the number of neutrophils treated with TNF-α, there was a significant difference in the number of adhered neutrophils (1-way ANOVA, Tukey's test).

### Example 3

### Human SAP Inhibition of Murine Neutrophil Adhesion Induced by TNF-α

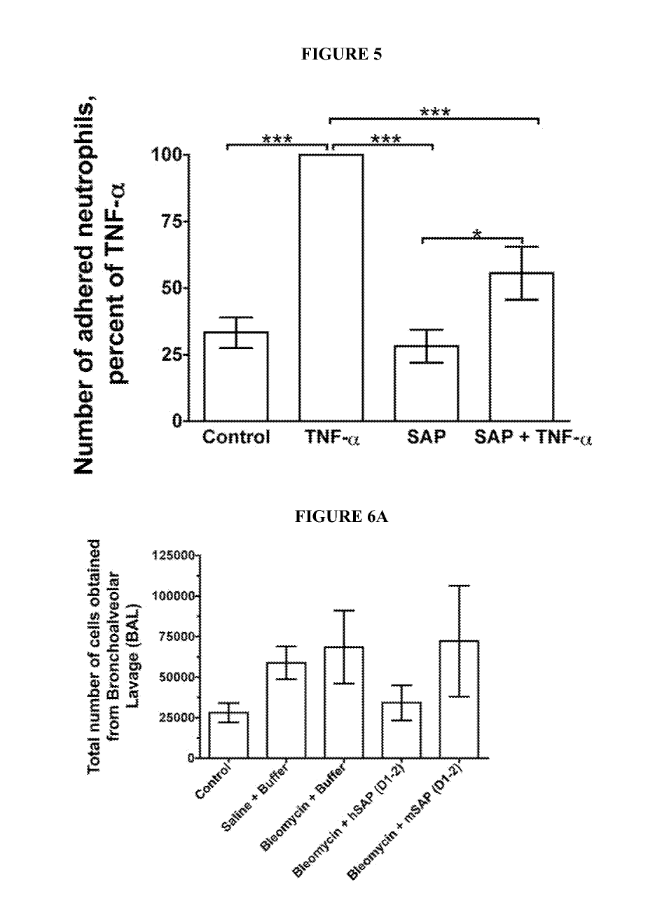

96-well tissue culture plates were pre-coated with 20 μg/ml plasma fibronectin and then blocked with 2% BSA-PBS for 2 hours at room temperature. Murine blood was lysed with ACK lysis buffer and isolated cells were incubated in the presence or absence of 60 μg/ml SAP for 30 minutes at 37° C. Cells were then plated on a 96-well tissue culture plate in the presence or absence of 100 ng/ml TNF-α for 30 minutes at 37° C. Non-adhered cells were removed, and adhered cells were stained for Ly6G (a marker for murine neutrophils). The number of adhered neutrophils was counted as described in Example 2 (n=8). As shown in FIG. 5, there was a significant difference in the number of adhered neutrophils (1-way ANOVA, Tukey's test).

### Example 4

### SAP Decreases the Accumulation of Ly6G-Positive Cells in 0.2 U/kg Bleomycin-Treated Mouse Lungs

Similar effects of human SAP on neutrophic adhesion using murine neutrophils instead of human neutrophils were confirmed (data not shown).

Mice were treated with 0.2 U/kg bleomycin using an oropharyngeal technique on day 0. Mice were then injected with either 50 μg SAP or an equal volume of buffer on days 1 and 2. After the mice were sacrificed on day 3, cells were collected by BAL.

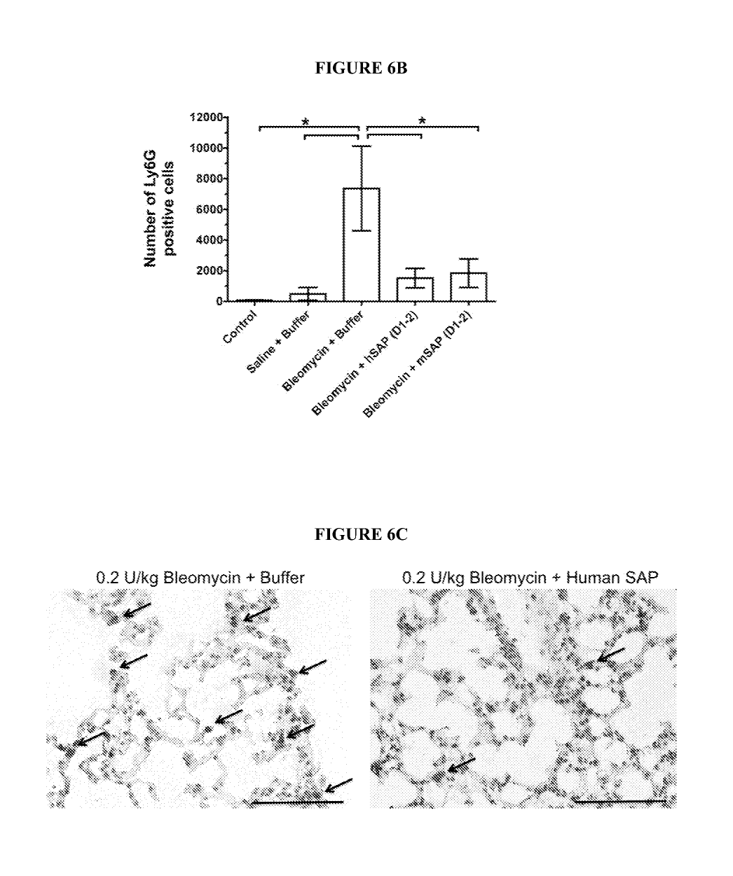

FIG. 6A shows the total number of cells collected from BAL of untreated mice (control), mice treated with saline and buffer, mice treated with 0.2 U/kg bleomycin and buffer, mice treated with 0.2 U/kg bleomycin and 50 μg hSAP, or mice treated with bleomycin and 50 μg mSAP. FIG. 6A further shows no significant difference between the number of cells gathered among any of the groups.

Using the non-parametric Mann Whitney two tailed t-test, there was an increased number of Ly6G-positive cells in the BAL from bleomycin and buffer when compared to control, mice treated with saline and buffer, or mice treated with bleomycin and human SAP, with * indicating p<0.05 as shown in FIG. 6B. Using the non-parametric Mann Whitney two tailed t-test, there was no significant difference between mice treated with bleomycin and buffer or mice treated with bleomycin and mouse SAP. However, using the non-parametric Mann Whitney one-tailed t-test, there was a statistically significant increase in the number of Ly6G-positive cells in the BAL from mice treated with bleomycin and buffer when compared to the number of Ly6G-positive cells in the BAL from mice treated with bleomycin and mouse SAP.

FIG. 6C illustrates images of sections from the lungs after performing BAL. After obtaining cells from BAL, day 3 lung sections from mice treated with 0.2 U/kg bleomycin and buffer or 0.2 U/kg bleomycin and human SAP were stained with anti-mouse Ly6G to detect neutrophils. Since the BAL removes non-adherent and poorly-adhered cells from the alveoli, the presence of a reduced number of neutrophils remaining in the lungs of animals treated with SAP indicates that if all of the lung neutrophils had been obtainable, the difference between bleomycin/buffer injections and bleomycin/SAP injections would have been even greater than the differences shown in FIG. 6B.

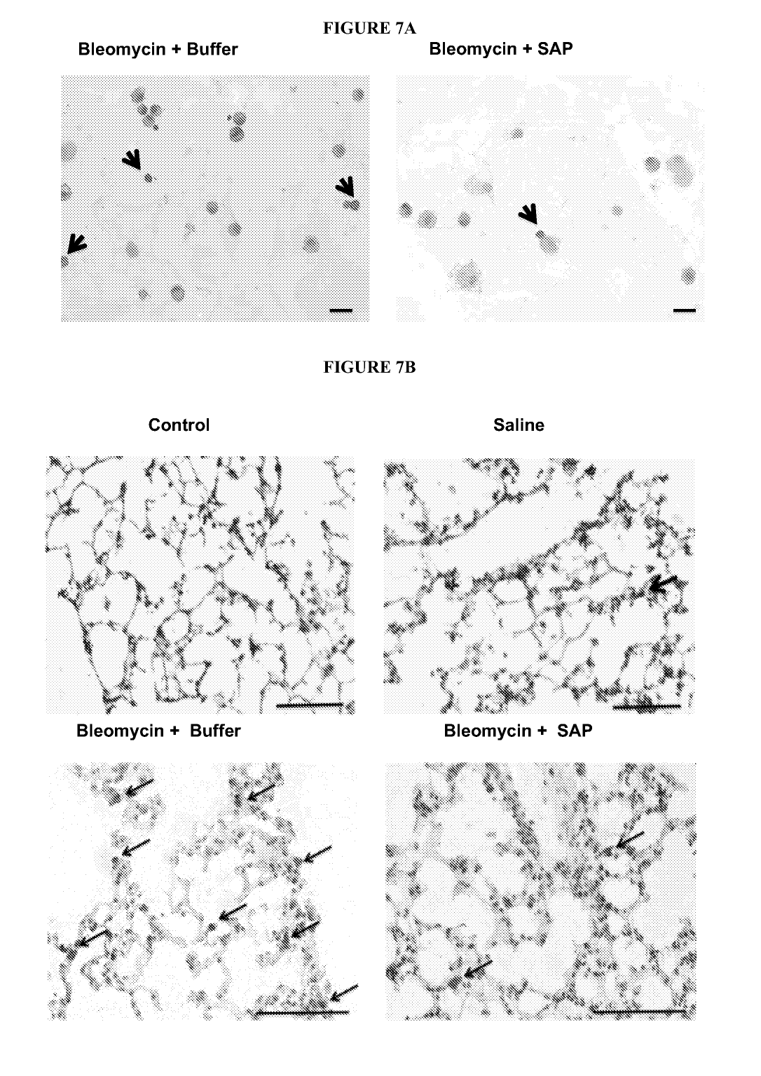

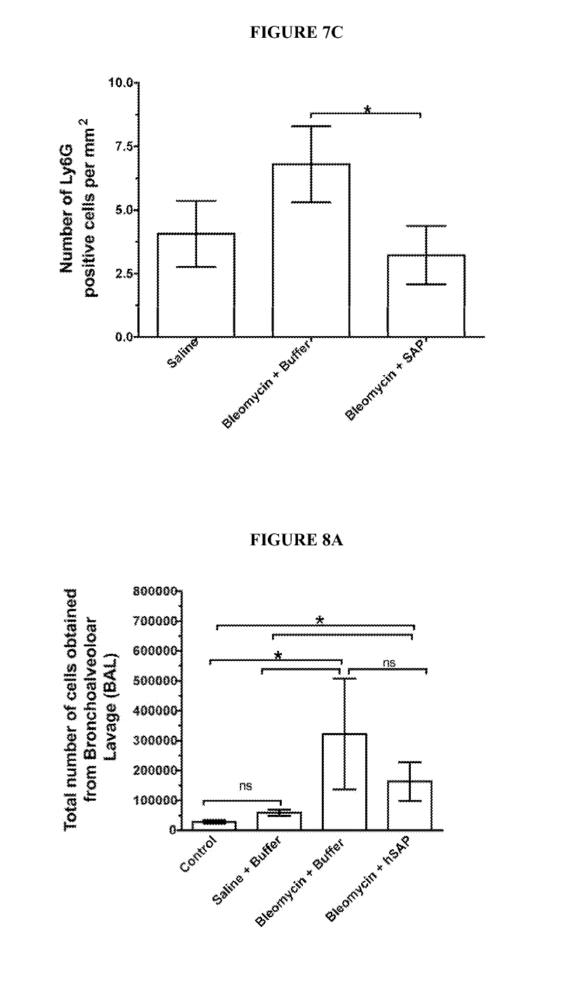

FIG. 7 shows results for the same experiment. FIG. 7A shows BAL cells stained for Ly6G. FIG. 7B illustrates images of sections from the lungs after performing BAL, with the sections stained for the neutrophil marker Ly6G. FIG. 7C shows counts of the Ly6G-positive cells in the lung sections. Compared to the lungs of mice treated with bleomycin and buffer, there was a statistically significant decrease in the number of neutrophils in the lungs after BAL in the mice treated with bleomycin and then treated with SAP.

### Example 5

### SAP Decreases the Accumulation of Ly6G-Positive Cells in the Lungs of 3 U/kg Bleomycin-Treated Mouse Lungs

Mice were treated with 3 U/kg bleomycin using an oropharyngeal technique on day 0. Mice were then injected with either 50 μg SAP or an equal volume of buffer on days 1 and 2. After the mice were sacrificed on day 3, cells were collected by BAL.

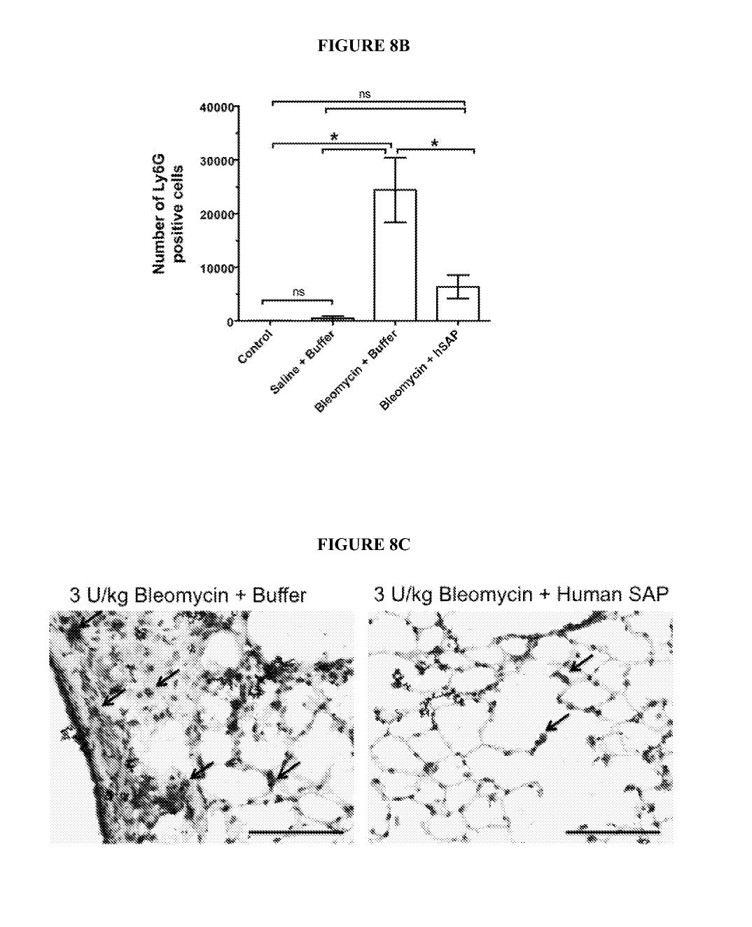

FIG. 8A illustrates the total number of cells collected from BAL of untreated mice (control), mice treated with saline and buffer, mice treated with 3 U/kg bleomycin and buffer, or mice treated with 3 U/kg bleomycin and 50 μg hSAP. As illustrated in FIG. 8A, there was no statistically significant difference between the total number of cells collected from the BAL of control or saline-treated mice. However, there was an increased number of total cells collected in the BAL from mice treated with 3 U/kg bleomycin and buffer or 3 U/kg bleomycin and human SAP when compared to the total cells collected in the BAL from control or saline-treated mice.

FIG. 8B illustrates the total number of Ly6G-positive cells in the above experimental groups. This shows that SAP injections decrease neutrophil influx into the lungs after bleomycin treatment.

FIG. 8C illustrates images of lung sections. After obtaining cells from BAL, day 3 lung sections from mice treated with 3 U/kg bleomycin and buffer or 3 U/kg bleomycin and human SAP were stained with anti-mouse Ly6G to detect neutrophils. Since the BAL removes non-adherent and poorly-adhered cells from the alveoli, the presence of a reduced number of neutrophils remaining in the lungs of animals treated with SAP indicates that if all of the lung neutrophils had been obtainable, the difference between bleomycin/buffer injections and bleomycin/SAP injections would have been even greater than the differences shown in FIG. 8B.

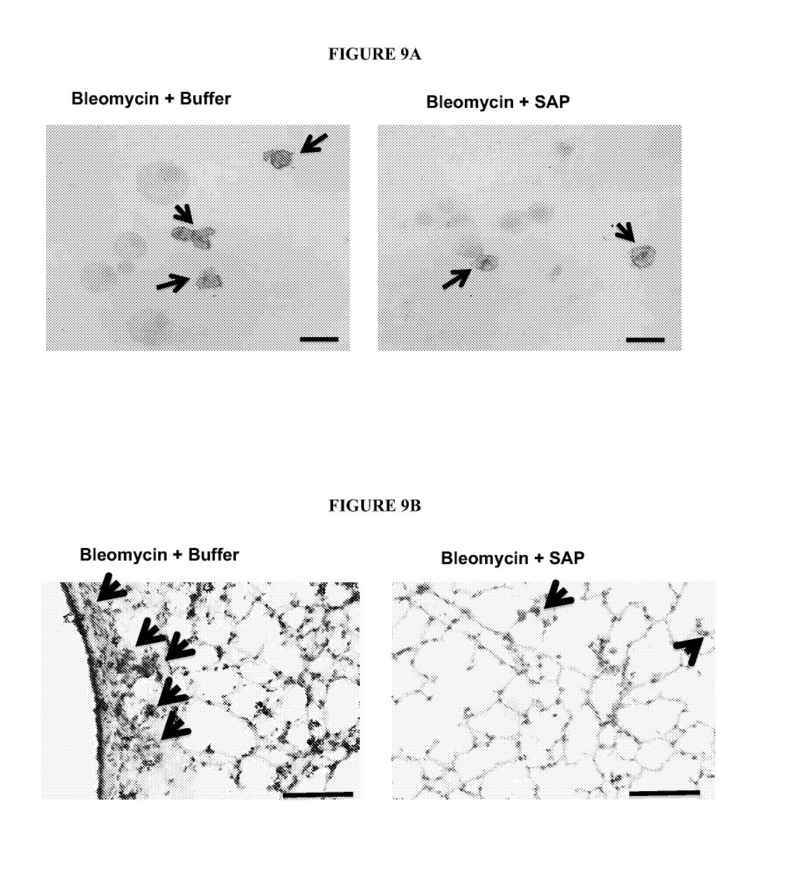

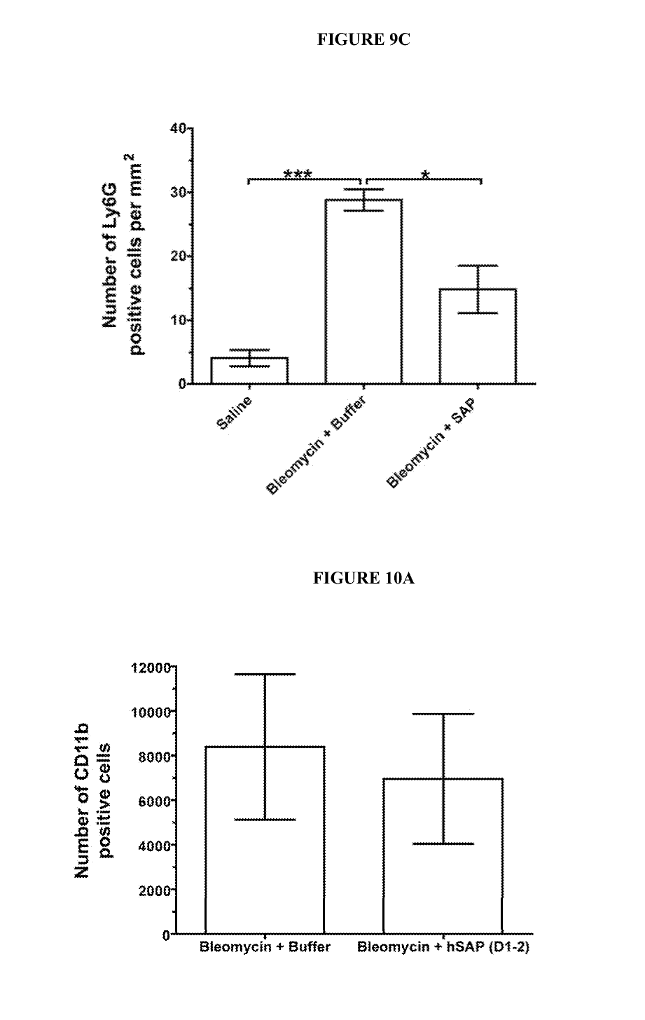

FIG. 9 shows results for the same experiment. FIG. 9A shows cells from the BAL stained for the neutrophil marker Ly6G. FIG. 9B illustrates images of lung sections. FIG. 9C shows counts of the Ly6G-positive cells in the lung sections. Compared to the lungs of mice treated with bleomycin and buffer, there was a statistically significant decrease in the number of neutrophils in the lungs after BAL in the mice treated with bleomycin and then treated with SAP.

### Example 6

### Macrophage and Leukocyte Assays

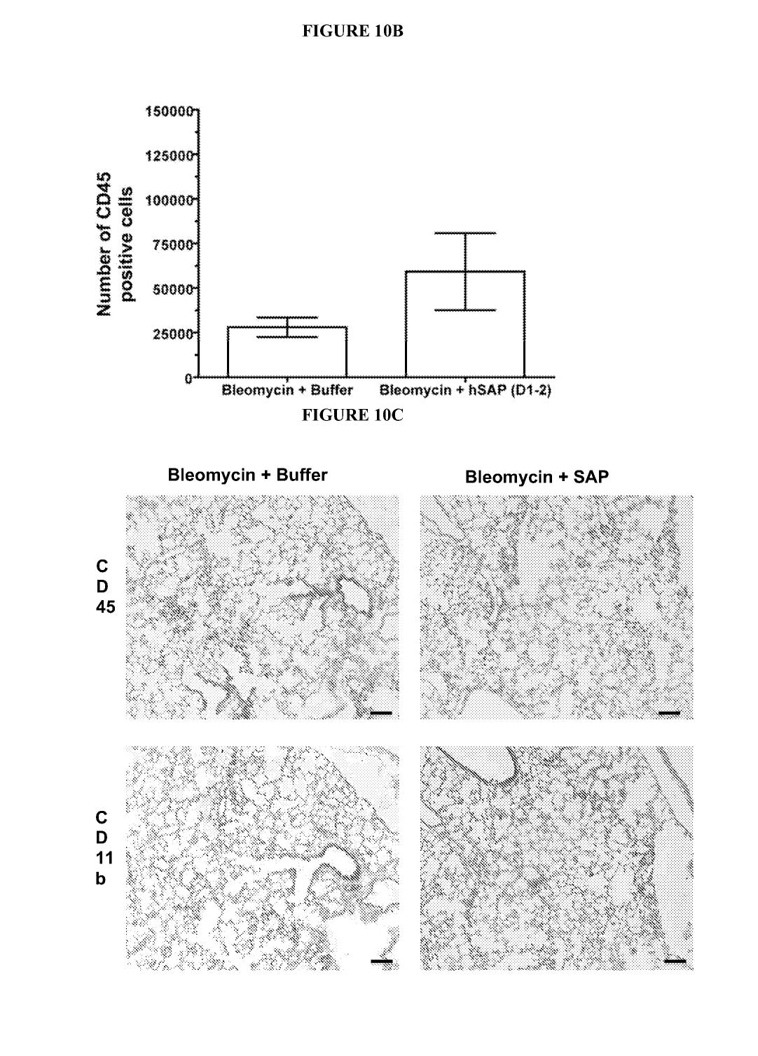

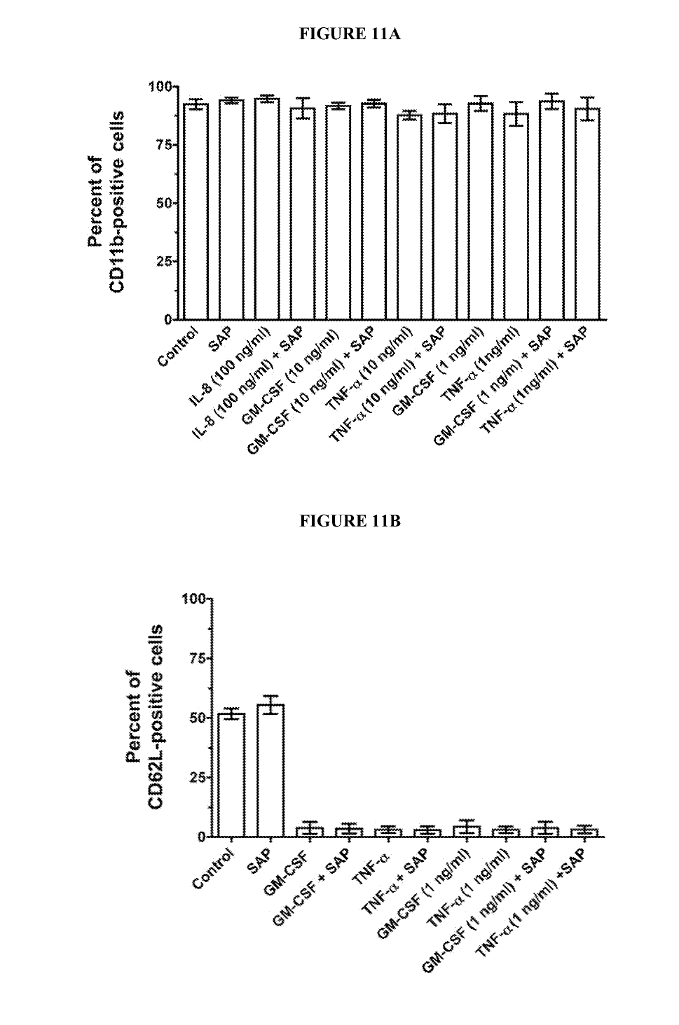

The lung sections from Examples 5 and 6 above were analyzed for the presence of macrophages and leukocytes using anti-CD11b (murine macrophage marker) and anti-CD45 antibodies. Results are shown in FIG. 10. No difference in CD11b-positive and CD45-positive cells was observed in the lung sections from mice treated with 0.2 U/kg bleomycin and buffer or mice treated with 0.2 U/kg bleomycin and human SAP. This suggests that, at 3 days, SAP has no effect on bleomycin-induced accumulation of macrophages or other leukocytes in the lungs.

### Example 7

### Staining for Neutrophil Adhesion Molecules

500 μl of neutrophils at 2.0×106 cells/ml were aliquoted into tubes (pre-coated with 2% BSA-RPMI for 1 hour at 37° C.) and incubated with 10 ng/ml or 1 ng/ml TNF-α, 100 ng/ml IL-8, or 10 ng/ml or 1 ng/ml GM-CSF in the presence or absence of 10 μg/ml or 60 μg/ml SAP for one hour at 37° C. For the neutrophils that were stained with (anti-human) anti-CD18, anti-CD61, or anti-CD44, SAP was added to 30 μg/ml. Cells were then washed with ice-cold PBS, collected by centrifugation at 500×g for 5 minutes, and resuspended in 1 ml of 4% BSA-PBS. Cells were stained in BSA-coated tubes with 5 μg/ml antibodies against CD11b (Biolegend), CD62L (BD Biosciences), CD32 (BD Biosciences), CD18 (Biolegend), CD61 (BD Biosciences), CD44 (BD Biosciences), or mouse IgG1 isotype control (Biolegend) for 30 minutes at 4° C. The cells were then washed three times in ice-cold PBS, and incubated with 2.5 μg/m1 FITC-conjugated F(ab′)2 goat anti-mouse IgG antibodies (cross-adsorbed against human Ig, Southern Biotechnology, Birmingham, Ala., USA) as described previously. The cells were washed three times in ice-cold PBS, resuspended in 200 μl 4% BSA-PBS, and analyzed by flow cytometry.

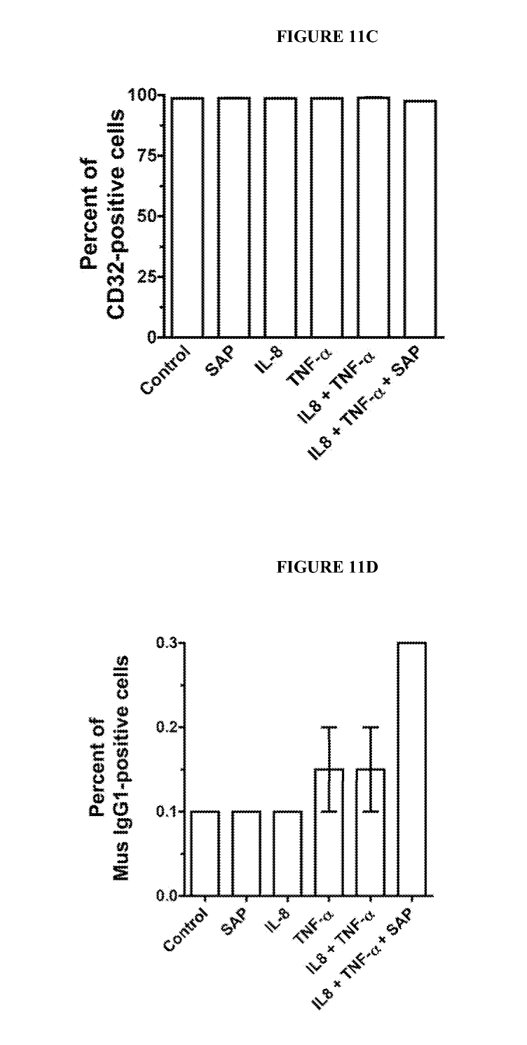

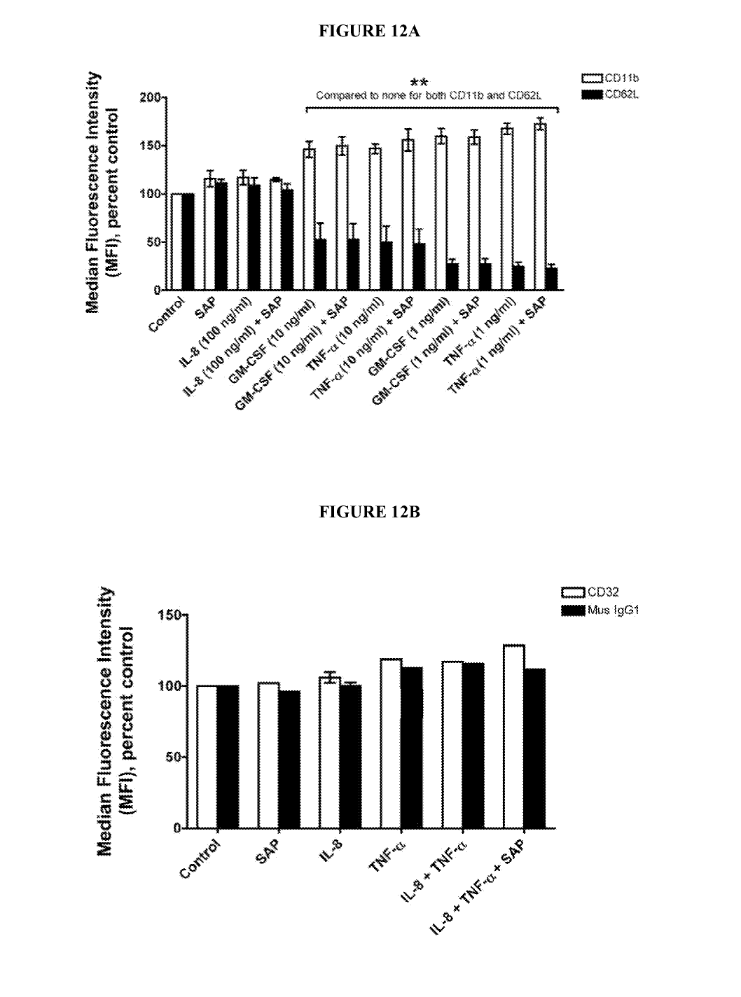

Levels of CD11b and CD62L typically change in activated neutrophils. CD32 (FCγRII) levels do not and thus that molecule served as a control. No treatment showed any significant effect on the number of CD11b-positive cells (FIG. 11A). IL-8 in the presence or absence of SAP had no effect on the levels of CD11b or CD62L compared to untreated neutrophils (FIG. 12A). TNF-α and GM-CSF induced increased levels of CD11b, but SAP had not effect on the activation (FIG. 12A). TNF-α and GM-CSF decreased the number and the levels of CD62L-positive cells, but SAP had no effect on the basal or stimulated CD62L levels (FIGS. 11B and 12A). There was no significant effect on the levels of CD32 or control mouse IgG1 staining when neutrophils were treated with TNF-α or IL-8 in the presence or absence of SAP (FIGS. 11C, 11D and 12B). Together, the data indicate that although TNF-α and GM-CSF alter levels of CD11b and CD62L on neutrophils, the addition of SAP has no obvious effect on the levels of these adhesion molecules or CD32.

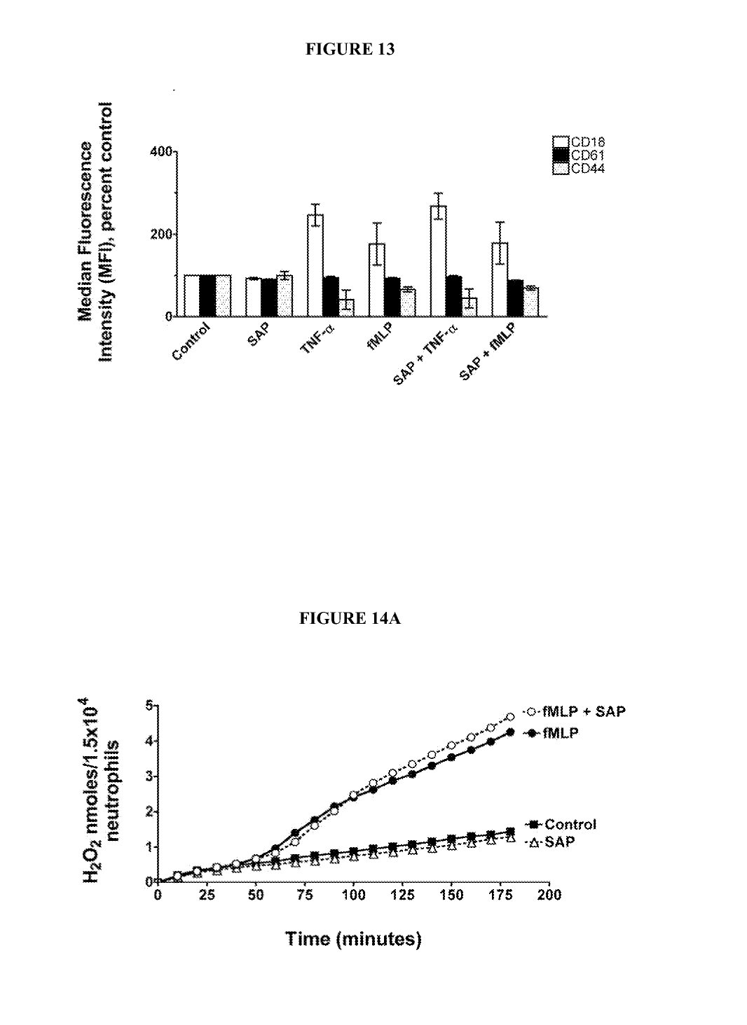

The effect of SAP on other adhesion molecules in neutrophils also showed no effect. TNF-α increased the levels of CD18 and decreased levels of CD44 on neutrophils, but had no effect on levels of CD61 (FIG. 13). Similarly, fMLP slightly increased levels of CD18 but had no significant effect on levels of CD61 or CD 44 (FIG. 13). SAP had no effect on the basal or stimulated levels of CD18, CD44, or CD61 (FIG. 13).

This suggests that SAP's effects on neutrophils do not occur through an effect on cell-surface levels of any of these adhesion molecules.

### Example 8

### Detection of Hydrogen Peroxide Production

Wells of black 96-well cell culture plates were pre-coated with 50 μl of 20 μg/ml plasma fibronectin for 1 hour at 37° C. The fibronectin was then removed, and the wells were washed three times with 200 μl of PBS, and then washed once with Krebs-Ringer phosphate glucose buffer (KRPG) (145 mM NaCl, 4.9 mM KCl, 0.54 mM CaCl2, 1.2 mM MgSO₄, 5.8 mM sodium phosphate, and 5.5 mM glucose, pH 7.35). 500 μl of neutrophils at 1.5×10⁶ cells/ml in KRPG were incubated in a tube (pre-incubated with 2% BSA-KRPG for 2 hours at 37° C.) and SAP was added to a final concentration of 30 μg/ml. As a control, a similar tube had an equal volume of buffer added to it. These were incubated for 30 minutes at 37° C. An assay mixture of 100 μl of KRPG, 20 μl of 300 μM scopoletin (Sigma) in KRPG, 20 μl of 10 mM NaN₃ in KRPG, and 20 μl of 10 U/ml horseradish peroxidase (Sigma) in KRPG were aliquoted into a well and the plate was equilibrated to 37° C. for 5 minutes as described previously. 20 μl of neutrophils incubated with or without 30 μg/ml SAP was then added to the assay mixture in the presence or absence of 20 μl of 1 μg/m1 TNF-α in KRPG, 20 μl of 1 μM formyl-Met-Leu-Phe (fMLP) (Sigma) in KRPG, 20 μl of 1 μM phorbol 12-myristate 13-acetate (PMA) (Sigma) in KRPG, 20 μl of 1 μM phorbol 12,13-dibutyrate (PDBu) (Sigma) in KRPG, or 20 μl of KRPG. The 96-well plate was incubated at 37° C. and the fluorescence (excitation: 360 nm emission: 460 nm) was monitored every 10 minutes for 3 hours using a Synergy MX plate reader (BioTek, Winooski, Vt.).

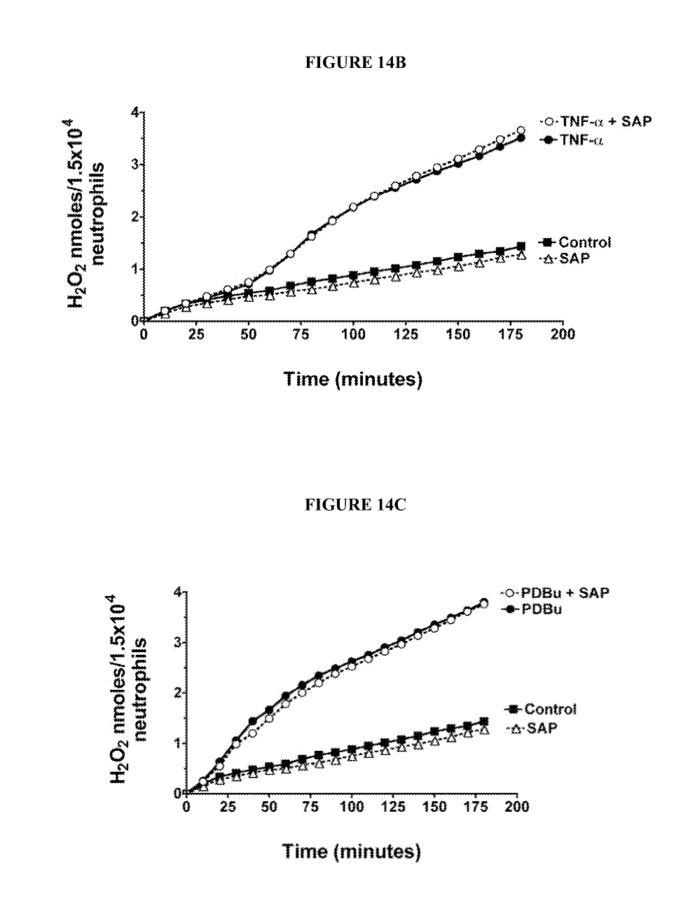

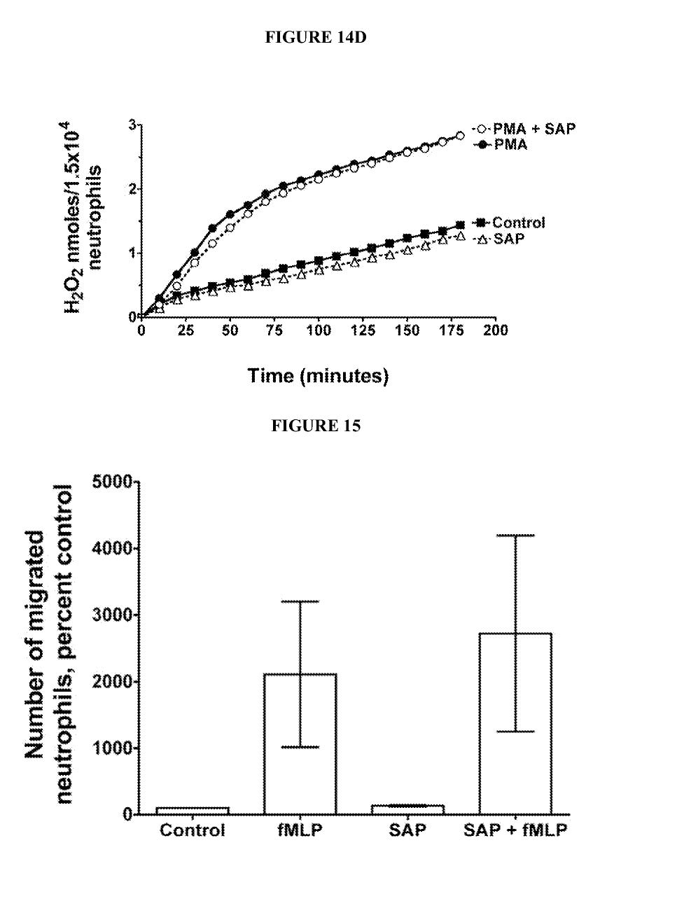

Activated neutrophils produce hydrogen peroxide, thus SAP's effects on hydrogen peroxide production was tested. Changes in hydrogen peroxide production were measured by measuring intensity of scopoletin, a fluorescent molecule modified by hydrogen peroxide. The production of hydrogen peroxide in cells treated with TNF-α, fMLP, PDBu, or PMA exceeded the production of hydrogen peroxide in control cells (FIG. 14). SAP had no significant effect on the production of hydrogen peroxide induced by fMLP (FIG. 14A), TNF-α (FIG. 14B), PDBu (FIG. 14C), or PMA (FIG. 14D). Together, these data indicate that although SAP affected neutrophil adhesion, it does not appear to affect neutrophil hydrogen peroxide production.

### Example 9

### Transmigration of Neutrophils

The formyl peptide fMLP induces migration of neutrophils. To determine the effect of SAP on neutrophyl migration induced by fMLP, Boyden chamber assays were conducted. 50 μl of neutrophils at 1×10⁶ cells/ml in 2% BSA-RPMI was added to the top chamber of a 3 μm pore size nylon membrane insert in a 24 well plate (BD) in the presence or absence of 10 nM fMLP, 30 μg/ml SAP, 10 nM fMLP and 30 μg/ml SAP or an equal volume of buffer in 2% BSA-RPMI. The bottom chambers contained 600 μl of 10 nM fMLP in 2% BSA-RPMI, 600 μl of 30 μg/ml SAP in 2% BSA-RPMI, 600 μl of 10 nM fMLP and 30 μg/ml SAP in 2% BSA-RPMI, or equal volumes of buffer in 2% BSA-RPMI. The transmigration was carried out for 2 hours at 37° C. The top chamber was removed, and the neutrophils that had migrated into the bottom chamber were then counted with a flow cytometer.

fMLP significantly increased the number of neutrophils that migrated across the porous membrane of the Boyden chamber, while SAP had no effect on the migration of neutrophils in the absence of fMLP or on the migration of neutrophils in the presence of fMLP (FIG. 15).

### Example 10

### Apoptosis of Neutrophils

Apoptosis of neutrophils may be delayed by certain compounds that otherwise affect their activity, such as GM-CSF. Accordingly, the effects of SAP on neutrophils were tested by measuring levels of annexin V, an indicator of apoptosis. 500 μl of neutrophils at 2.0×10⁶ cells/ml were aliquoted into tubes (pre-coated with 2% BSA-RPMI for 1 hour at 37° C.) and incubated with 10 ng/ml or 1 ng/ml TNF-a, or 10 ng/ml or 1 ng/ml GM-CSF in the presence or absence of 60 μg/ml SAP for 22 hours at 37° C. The cells were then washed with ice-cold PBS, collected by centrifugation at 500×g for 5 minutes, and re-suspended in 1 ml of 4% BSA-PBS. Cells were stained with 5 μg/ml Alexafluor 488-conjugated annexin V (Invitrogen) for 30 minutes at 4° C. The cells were then washed three times in ice-cold PBS, resuspended in 200 μl 4% BSA-PBS, and analyzed with a flow cytometer.

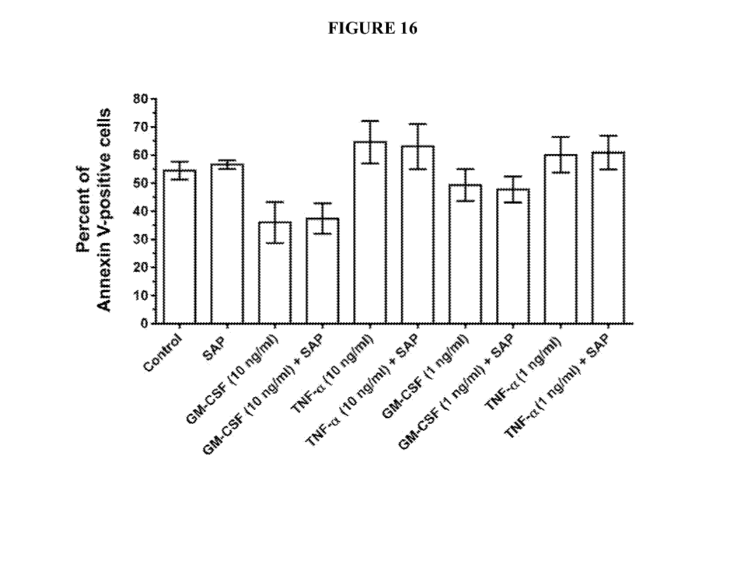

Neutrophils treated with TNF-α or GM-CSF in the presence or absence of SAP showed a difference in annexin V levels only in response to GM-CSF, which decreased the percentage of annexin V-positive cells, consistent with its ability of inhibit apoptosis (FIG. 16). Thus SAP does not appear to affect neutrophil apoptosis.

Therefore, the present invention is well adapted to attach the ends and advantages mentioned as well as those that are inherent therein. Although only exemplary embodiments of the disclosure are specifically described above, it will be appreciated that modifications and variations of these examples are possible without departing from the spirit and intended scope of the disclosure. For example, although the effects of SAP on neutrophils are specifically described herein, similar effects may be seen on other granulocytes, such as eosinophils and basophils, and may confer similar benefits. While compositions and methods are described in terms of “comprising,” “containing,” or “including” various components or steps, the compositions and methods can also “consist essentially of or ” consist of the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. Further, the term “or” as used herein is intended to be inclusive, not exclusive, unless an exclusive meaning is required by context. If there is any conflict in the usages of a word or term in this specification and one or more documents that may be incorporated herein by reference, the definitions that are consistent with this specification should be adopted.

1-50. (canceled)

51. A method of reducing the number of neutrophils in a body region comprising administering a serum amyloid P (SAP) composition in an amount and for a time sufficient to suppress neutrophil movement into the body region.

52. The method according to claim 51, wherein the SAP comprises a portion of full-length mammalian SAP.

53. The method according to claim 51, wherein the SAP composition comprises a biodegradable polymer bound to the SAP.

54. The method according to claim 51, wherein the body region comprises the lungs and administering comprises administering by inhalation.

55. The method according to claim 51, wherein administering comprises injection.

56. The method according to claim 51, wherein administering comprises topical administration.

57. The method according to claim 51, wherein the SAP is administered within 30 minutes, within 45 minutes, or within 60 minutes after an event triggering neutrophil entry into the body region.

58. The method according to claim 51, wherein the SAP is administered within 2 hours, within 4 hours, within 6 hours, or within 8 hours after an event triggering neutrophil entry into the body region.

59. The method according to claim 51, wherein the SAP is administered within 24 hours, within 48 hours, or within 72 hours after an event triggering neutrophil entry into the body region.

60. A method of reducing the number of neutrophils in the lungs of a patient suffering from acute respiratory distress syndrome (ARDS) comprising administering a serum amyloid P (SAP) composition to the patient in an amount and for a time sufficient to suppress neutrophil movement into the lungs.

61. The method according to claim 60, wherein the SAP comprises a portion of full-length mammalian SAP.

62. The method according to claim 60, wherein the SAP composition comprises a biodegradable polymer bound to the SAP.

63. The method according to claim 60, wherein administering comprises administering by inhalation.

64. The method according to claim 60, wherein the SAP is administered within 30 minutes, within 45 minutes, or within 60 minutes after an event triggering ARDS.

65. The method according to claim 60, wherein the SAP is administered within 2 hours, within 4 hours, within 6 hours, or within 8 hours after an event triggering ARDS.

66. The method according to claim 60, wherein the SAP is administered within 24 hours, within 48 hours, or within 72 hours after an event triggering ARDS.

67. A method of treating a disease, wherein at least one of the indicators of the disease is the number of neutrophils in a body region of a patient, comprising administering a serum amyloid P (SAP) composition to the patient in an amount and for a time sufficient to suppress neutrophil movement into the body region or enhancing neutrophil movement out of the body region.
